(19) United States
(12) Patent Application Publication
(10) Pub. No.: US 2022/0273614 A1
(43) Pub. Date: Sep. 1, 2022

(54) INTRAVENOUS VITAMIN C THERAPY PROTOCOL FOR THE TREATMENT OF CANCER

(71) Applicants: Ronald HUNNINGHAKE, Wichita, KS (US); Thomas LEVY, Wichita, KS (US); Lucas TIMS, Wichita, KS (US); Christopher BRANNON, Wichita, KS (US); Riordan Clinic, Wichita, KS (US)

(72) Inventors: Ronald HUNNINGHAKE, Wichita, KS (US); Thomas Levy, Wichita, KS (US); Lucas Tims, Wichita, KS (US); Christopher Brannon, Wichita, KS (US)

(21) Appl. No.: 17/631,833

(22) PCT Filed: Jul. 31, 2020
(86) PCT No.: PCT/US2020/044607

§ 371 Date: Jan. 31, 2022

Related U.S. Application Data
(60) Provisional application No. 62/881,294, filed on Jul. 31, 2019.

(51) Int. Cl. A61K 31/375; A61K 9/00; A61P 35/00

(52) U.S. Cl. CPC A61K 31/375; A61K 9/0019; A61P 35/00

(57) ABSTRACT

Provided herein are methods for treating cancer or chronic illness in a subject including administering an intermittent, high dose of vitamin C and subsequently administering a continuous perfusion of a lower dose of vitamin C.

### CROSS-REFERENCED APPLICATIONS

This application claims priority to U.S. Provisional Application No. 62/882,294, filed Jul. 31, 2019, the entirety of which is incorporated by reference herein.

### BACKGROUND

High-dose vitamin C has been used as a chemotherapeutic and biological response modifying agent. Research has shown that vitamin C, when infused intravenously is selectively toxic to cancer cells without harming normal cells.

Research to date provides the backbone to safely administer high-dose vitamin C infusions as a therapy and adjunct therapy in the treatment of cancer and chronic conditions.

There exists a need for cancer treatment methods that have effect on the tumor regression, improve the efficacy of the immune system, decrease side effects of chemo/radiation therapy and reduce or eliminate development of resistance to the anticancer therapies.

### BRIEF SUMMARY

Conventional chemotherapy is plagued with a phenomenon called “chemo-resistance.” The initial dosing of powerful chemotherapeutic agents is often effective at causing the tumor to shrink as large numbers of the cancer cells are killed. During the pause between chemo infusions, the cancer cells that weren't killed can re-proliferate. This results in a “survival of the fittest” or natural selection where regrowth of new, stronger tumor cells is then able to resist further chemo treatments. Unfortunately, this same phenomenon can occur with intermittent, high-dose IVC treatment of cancer. High dose IVC given twice a week may initially show great results, only to have the recipient beset with cancer recurrence or faster progression later on.

In view of the foregoing, there is a need for nontoxic and adjunctive medical treatment to address chronic illness and cancer in a manner that reduces tumor associated markers and/or inhibits tumor resistance. The present disclosure addresses this need, and provides additional benefits as well.

Provided herein are methods for potential benefits on the tumor response, improvement of the survival, providing better quality of life and reducing chemotherapeutic resistance in a cancer patient including administering a pulse dose of vitamin C and subsequently administering a continuous perfusion of a lower dose of vitamin C, where the continuous perfusion is administered over an infusion window.

Provided herein are methods for treating cancer in a subject having cancer including administering a pulse dose of vitamin C and subsequently administering a continuous perfusion of a lower dose of vitamin C.

Provided herein are methods for treating cancer in a subject having cancer including administering a high dose of vitamin C where the high dose includes from about 15 grams to about 50 grams vitamin C, every other day and subsequent to each high dose, administering a continuous perfusion of a lower dose of vitamin C where the lower dose is administered by continuous perfusion. For example, the lower dose may be administered over an infusion window of about 8 to about 48 hours at a rate of about 0.5 grams vitamin C per hour.

### BRIEF DESCRIPTION OF THE DRAWINGS

FIG. 1 shows ascorbate concentration (mM) in plasma before and during intravenous vitamin C (IVC) treatment. Pie chart insert shows number of patients with low (Lo), high (Hi), or normal (Norm) ascorbate levels before treatment.

FIG. 2 shows plasma ascorbate concentrations (mM) during intravenous vitamin C (IVC) therapy for several patients given a dose 150 mg/kg/day-710 mg/kg/day by continuous infusion.

FIGS. 3A-C show percentage of change in absolute neutrophil counts (shown in FIG. 3A), percentage of change absolute lymphocyte count vs. pre-treatment levels (shown in FIG. 3B) and absolute values of ALCs before and after intervention for patients with ALCs lower than normal range (shown in FIG. 3C). Different dosages in FIG. 3(A, B) are indicated by the different shapes. Spearmen's rank correlation coefficient, non-parametric p-values, and regression lines are given.

FIGS. 4A-B demonstrate rate of change in the neutrophil-to-lymphocyte ratio (ΔNLR) for each patient before and after therapy. FIG. 4A shows ΔNLR values pre-IVC and at the end IVC therapy (post). FIG. 4B shows correlation between patient survival time (days after start of therapy) and the ΔNLR value at the end of therapy.

FIG. 5 shows survival times (days) of cancer patients with normal (LDH<245 U/L) or above normal range (NR) (LDH>245 U/L) LDH concentrations.

FIG. 6 shows survival time for patients with a greater than 20% decrease in creatinine levels versus those with a less than 20% decrease.

FIGS. 7A-7B demonstrate changes in blood glucose during intravenous vitamin C (IVC) therapy for patients with the highest blood glucose levels. FIG. 7A shows change in plasma glucose concentrations (mg/dL) with time of IVC therapy for several cancer patients (time zero represent the onset of therapy). FIG. 7B shows percentage of plasma glucose concentration change during IVC therapy as function of pre-treatment glucose concentrations (mg/dL). Spearman's rank correlation coefficient and the p-value for significance are given. The line represents the second order polynomial extrapolation of the data.

FIG. 8 is a graph of adverse events reported for patients given intravenous vitamin C (IVC) infusion therapy.

### DETAILED DESCRIPTION

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the terms “disease” or “condition” refer to a state of being or health status of a patient or subject capable of being diagnosed and/or treated with compounds or methods provided herein. The disease may be a cancer.

As used herein, the term “cancer” refers to all types of cancer, neoplasm or malignant tumors found in mammals (e.g. humans), including leukemias, lymphomas, carcinomas and sarcomas. Examples of cancers that may be diagnosed and/or treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Example cancers that may be diagnosed and/or treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, the term “diagnosis” refers to an identification or likelihood of the presence of a particular type of cancer or outcome in a subject. As also used herein, the term “prognosis” refers to the likelihood or risk of a subject developing a particular outcome or particular event.

As used herein, the terms “treating”, or “treatment” refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

As used herein, the term “prevent” refers to a decrease in the occurrence of disease symptoms in a patient. The prevention may be complete (no detectable symptoms) or partial, such that fewer symptoms are observed than would likely occur absent treatment.

As used herein, the terms “patient” and “subject” refer to a living organism suffering from or prone to a disease or condition that can be treated by administration of a pharmaceutical composition. Non-limiting examples include humans, other mammals, bovines, rats, mice, dogs, monkeys, goat, sheep, cows, deer, and other non-mammalian animals. In some embodiments, a subject is human.

As used herein, the terms “control” and “control experiment” are used in accordance with their plain and ordinary meaning and refer to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is a measurement of a reference sample (e.g., non-cancer cells or untreated cancer cells) as described herein (including embodiments and examples).

As described herein, the terms “marker,” “protein marker,” “polypeptide marker,” and “biomarker” are used interchangeably throughout the disclosure. As used herein, a protein marker refers generally to a protein or polypeptide, the level or concentration of which is associated with a particular biological state, particularly a state associated with cancer, tumor, a specific cancer or a specific tumor.

As described herein, the term “effective amount” refers to an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. The exact amounts will depend on the purpose of the treatment, and will be ascertainable by one skilled in the art using known techniques.

For any compound described herein, the therapeutically effective amount can be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

As described herein, the term “therapeutically effective amount” refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As described herein, the term “administering” refers to oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent. In embodiments, administration is intravenous administration. In embodiments, administration is continuous perfusion.

As described herein, the term “co-administer” refers to a composition described herein administered at the same time, just prior to, or just after the administration of one or more additional therapies. The compounds provided herein can be administered alone or can be coadministered to the patient. Co-administration is meant to include simultaneous or sequential administration of the compounds individually or in combination (more than one compound). Thus, the preparations can also be combined, when desired, with other active substances (e.g. to inhibit or kill cancer cells).

As defined herein, the terms “activation,” “activate,” “activating,” “activator” and the like are used in accordance with its plain ordinary meaning and refer to an interaction that positively affects (e.g. increasing) the activity or function of a protein or cell relative to the activity or function of the protein or cell in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein that is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

As used herein, the terms “agonist,” “activator,” “upregulator,” etc. are used in accordance with its plain ordinary meaning and refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As used herein, the terms “inhibition,” “inhibit,” “inhibiting” and the like are used in accordance with its plain ordinary meaning and refer to an interaction that negatively affecting (e.g. decreasing) the activity or function of the protein or cell relative to the activity or function of the protein or cell in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein).

As used herein, the terms “inhibitor,” “repressor” or “antagonist” or “downregulator” are used in accordance with its plain ordinary meaning and refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

As described herein, the term “anticancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rIL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like

As used herein, the terms “vitamin C,” “ascorbic acid” and “L-ascorbic acid” refer to a vitamin found in various foods and sold as a dietary supplement. Vitamin C is an essential nutrient for certain animals including humans. The term vitamin C encompasses several vitamers that have vitamin C activity in animals. Ascorbate salts such as sodium ascorbate and calcium ascorbate are used in some dietary supplements. These release ascorbate upon digestion. Ascorbate and ascorbic acid are both naturally present in the body, since the forms interconvert according to pH. Oxidized forms of the molecule such as dehydroascorbic acid are converted back to ascorbic acid by reducing agents. Vitamin C functions as a cofactor in many enzymatic reactions in animals (and humans) that mediate a variety of essential biological functions, including wound healing and collagen synthesis. Another biochemical role of vitamin C is to act as an antioxidant (a reducing agent) by donating electrons to various enzymatic and non-enzymatic reactions. Doing so converts vitamin C to an oxidized state—either as semi dehydroascorbic acid or dehydroascorbic acid. These compounds can be restored to a reduced state by glutathione and NADPH-dependent enzymatic mechanisms.

As used herein, the term “lymphocyte” refers to one of the subtypes of a white blood cell in a vertebrate's immune system. Lymphocytes include natural killer cells (which function in cell-mediated, cytotoxic innate immunity), T cells (for cell-mediated, cytotoxic adaptive immunity), and B cells (for humoral, antibody-driven adaptive immunity). They are the main type of cell found in lymph. The three major types of lymphocyte are T cells, B cells and natural killer (NK) cells. Lymphocytes can be identified by their large nucleus.

As used herein, the term “neutrophil” also known as “neutrocytes” refers to the most abundant type of granulocytes and the most abundant (60% to 70%) type of white blood cells in most mammals. They form an essential part of the innate immune system. Their functions vary in different animals. They are formed from stem cells in the bone marrow and differentiated into subpopulations of neutrophil-killers and neutrophil-cagers. They are short-lived and highly mobile as they can enter parts of tissue where other cells/molecules cannot. Neutrophils may be subdivided into segmented neutrophils and banded neutrophils (or bands). They form part of the polymorphonuclear cells family (PMNs) together with basophils and eosinophil.

As used herein, the term “lactate dehydrogenase” or “LDH” refers to an enzyme found in nearly all living cells (animals, plants, and prokaryotes). LDH catalyzes the conversion of lactate to pyruvate and back, as it converts NAD⁺ to NADH and back.

As used herein, the term “creatinine” refers to a is a breakdown product of creatine phosphate in muscle, and is usually produced at a fairly constant rate by the body (depending on muscle mass). Serum creatinine (a blood measurement) is an important indicator of renal health because it is an easily measured byproduct of muscle metabolism that is excreted unchanged by the kidneys. Creatinine itself is produced via a biological system involving creatine, phosphocreatine (also known as creatine phosphate), and adenosine triphosphate (ATP, the body's immediate energy supply).

As used herein, the term “pulse” refers to an intermittent frequency of high-dose administration of a therapeutic composition. As used herein, “pulse” or a “pulsed approach” or a “pulse” dose refers to a high dose administration of intravenous vitamin C.

As used herein, the term “press” refers to continuous low to mid doses of vitamin C infusion. As used herein, “press” or “press approach” or “press dose” refers to a continuous administration of a low to mid-level dose intravenous vitamin C. Examples of methods for administering a press dose are described herein. In some embodiments, the press dose is administered over an 8 to 48 hour window.

As used herein, the term “intermittent” refers to a therapeutic frequency during which there are periods of administration between which there are periods of rest or no administration of therapy.

As used herein, the term “pump” refers any electric pump or ambulatory infusion pump, such as elastomeric pump, that is capable of IV infusing over an extended period of time.

As used herein, the term “elastomeric pump” refers to a device that infuses medication once the tubing is unclamped. Built with an elastic balloon inside a very tough outer cover, the device pushes intravenous medication through tubing and a filter that is attached to the reservoir.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Methods

In an aspect, provided herein are methods for improved administration of intravenous vitamin C. The methods can include, for example, administering the vitamin C according to one or more “pulse” doses and one or more “press” doses as described herein. In some embodiments, such a protocol can provide unexpected and surprising benefits compared to prior continuous infusion methods that do not pulse and/or press. For example, the instant methods can result in less toxicity, greater efficacy, ability to tolerate lower doses, and/or avoidance of resistance, for example.

In an aspect, provided herein are methods for affecting tumor progression, boosting the immune system, improving survival, providing better quality of life, and reducing the development of resistance to anticancer therapies. The methods include administering a pulse dose of vitamin C and subsequently administering a continuous perfusion of a lower dose of vitamin C, where the continuous perfusion is administered over an infusion window.

In an aspect, provided herein are methods for treating cancer in a subject having cancer including administering a pulse dose of vitamin C and subsequently administering a continuous perfusion of a lower dose of vitamin C.

In embodiments, the methods described herein include treating cancer. In embodiments, the cancer is a cancer selected from brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, Non-Hodgkin's Lymphomas, cancer of the thyroid, endocrine system, breast, cervix, colon, head & neck, liver, kidney, lung, ovary, pancreas, rectum, stomach, and uterus. In embodiments, the cancer is brain cancer. In embodiments, the cancer is glioma. In embodiments, the cancer is glioblastoma. In embodiments, the cancer is neuroblastoma. In embodiments, the cancer is prostate cancer. In embodiments, the cancer is colorectal cancer. In embodiments, the cancer is pancreatic cancer. In embodiments, the cancer is medulloblastoma. In embodiments, the cancer is melanoma. In embodiments, the cancer is cervical cancer. In embodiments, the cancer is gastric cancer. In embodiments, the cancer is ovarian cancer. In embodiments, the cancer is lung cancer. In embodiments, the cancer is cancer of the head. In embodiments, the cancer is Hodgkin's Disease. In embodiments, the cancer is Non-Hodgkin's Lymphoma. In embodiments, the cancer is of the thyroid. In embodiments, the cancer is endocrine cancer. In embodiments, the cancer is breast cancer. In embodiments, the cancer is cervical cancer. In embodiments, the cancer is colon cancer. In embodiments, the cancer is head & neck cancer. In embodiments, the cancer is liver cancer. In embodiments, the cancer is kidney cancer. In embodiments, the cancer is lung cancer. In embodiments, the cancer is ovarian cancer. In embodiments, the cancer is pancreatic cancer. In embodiments, the cancer is rectal cancer. In embodiments, the cancer is stomach cancer. In embodiments, the cancer is uterine cancer.

In some embodiments the methods described herein can be administered to an identified or selected person or patient. For example, the patient can be identified or selected based upon having an illness or disease as described herein (e.g., one of the cancers). The patient can be identified or selected based upon being susceptible to, having a need to avoid, having a resistant cancer, or having a cancer that has or develops resistance. The patient can be selected based upon having received a prior vitamin C treatment without the pulse and/or press methodologies described herein. The patient can be selected or identified based upon having leukocyte and neutrophil numbers that are below normal. The patient can be selected or identified based upon decreases in the rate of growth of neutrophil to lymphocyte ratio (NLR). The patient can be selected or identified based upon having an elevated lactate dehydrogenase (LDH) level compared to a reference and the method reduces lactate dehydrogenase (LDH) level An ongoing therapy also can be continued based upon the selection or identification criteria described above.

In some embodiments the therapy as described herein can be stopped if leukocyte and neutrophil numbers increase to normal, if the rate of growth of neutrophil to lymphocyte ratio (NLR) normalizes, if the lactate dehydrogenase (LDH) level decreases, stabilizes or normalizes, if a biomarker level improves showing an improvement in the cancer therapy (e.g., the marker goes down evidencing a reduction or improvement cancer, or the marker goes up showing a reduction or improvement in the cancer), if the cancer stabilizes,

In embodiments, the methods include a pulse dose administered over time of about 1 to about 2 hours. In embodiments, the methods include a pulse dose administered over time of about 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, or 120 minutes. In embodiments, the methods include a pulse dose administered over time of about 5 minutes-120 minutes, 10 minutes-115 minutes, 15 minutes-110 minutes, 20 minutes-105 minutes, 25 minutes-100 minutes, 30 minutes-95 minutes, 35 minutes-90 minutes, 40 minutes-85 minutes, 45 minutes-80 minutes, 50 minutes-75 minutes, or 60 minutes-70 minutes.

In embodiments, the method includes a pulse dose repeated at least once weekly. In embodiments, the method includes a pulse dose repeated at least twice weekly. In embodiments, the method includes a pulse dose repeated at least three times weekly. In embodiments, the method includes a pulse dose repeated once weekly. In embodiments, the method includes a pulse dose repeated a twice weekly. In embodiments, the method includes a pulse dose repeated three times weekly.

In embodiments, the methods include a pulse dose administered intravenously

In embodiments, the methods include a press dose administered in an infusion window of about 8 to about 48 hours. In embodiments, the methods include a press dose administered in an infusion window of about 10 to about 46 hours, about 12 to about 44 hours, about 14 to about 42 hours, about 16 to about 40 hours, about 18 to about 38 hours, about 20 to about 36 hours, about 22 to about 34 hours, about 24 to about 32 hours, or about 26 to about 30 hours.

In embodiments, the methods include continuous perfusion of a press dose that is administered using an elastomeric pump, portable intravenous infusion pump, ambulatory infusion pump, electric pump designed to infuse over extended periods of time and the like. In embodiments, the methods include continuous perfusion of a press dose that is administered using an elastomeric pump.

In embodiments, the pulse dose is between 15 grams and 100 grams of vitamin C. In embodiments, the pulse dose is between 20 and 95 grams, between 25 and 90 grams, between 30 and 85 grams, between 35 and 80 grams, between 40 and 75 grams, between 45 and 70 grams, or between 50 and 65 grams of vitamin C. In embodiments, the pulse dose is about 15, 20, 25, 30, 35, 40, 45, 50, 55, 60, 65, 70, 75, 80, 85, 90, 95, or 100 grams of vitamin C. In embodiments, the pulse dose is greater than 15, greater than 20, greater than 25, greater than 30, greater than 35, greater than 40, greater than 45, greater than 50, greater than 55, greater than 60, greater than 65, greater than 70, greater than 75, greater than 80, greater than 85, greater than 90, greater than 95, or greater than 100 grams of vitamin C.

In embodiments, the lower dose (press dose) is between 4 grams and 50 grams vitamin C per infusion window. In embodiments, the lower dose (press dose) is between 5 and 45 grams, between 10 and 40 grams, between 15 and 35 grams, or between 20 and 30 grams of vitamin C per infusion window In embodiments, the lower dose (or press dose) is about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 grams vitamin C per infusion window.

In embodiments, the lower dose (press dose) is administered at a perfusion rate of about 0.5 grams vitamin C per hour. In embodiments, the lower dose (press dose) is administered at a perfusion rate of about 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, or 1.00 grams vitamin C per hour.

In embodiments, the lower dose (press dose) is administered up to seven days per week. In embodiments, the lower dose (press dose) is administered up to one day, up to two days, up to three days, up to four days, up to five days, or up to six days per week. In embodiments, the lower dose (press dose) is administered one day, two days, three days, four days, 5 days, or 6 days per week.

In an aspect, provided herein are methods including administering a pulse dose of vitamin C and subsequently administering a continuous perfusion of a lower dose of vitamin C, wherein the continuous perfusion is administered over an infusion window.

In an aspect, provided herein are methods for treating cancer in a subject having cancer including administering a high dose of vitamin C every other day and subsequent to each high dose, administering a continuous perfusion of a lower dose of vitamin C by continuous perfusion over an infusion window.

In embodiments, methods described herein include administering a continuous perfusion of a lower dose of vitamin C, wherein the continuous perfusion is administered over an infusion window of about 8 to about 48 hours.

In embodiments, methods described herein include administering a pulse dose of vitamin C, where the pulse or high dose includes from about 15 grams to about 50 grams vitamin C. In embodiments, methods described herein include administering a continuous perfusion or press of a lower dose of vitamin C, where the lower dose is administered at a rate of about 0.5 grams vitamin C per hour.

In embodiments, the subject has an elevated lactate dehydrogenase (LDH) level compared to a reference and the method reduces lactate dehydrogenase (LDH) level. In embodiments, the reference is a control value. In embodiments, the reference is the lactate dehydrogenase (LDH) level in a sample from a healthy person.

In embodiments, the subject has leukocyte and neutrophil numbers are below normal compared to a reference and wherein the method increases leukocyte and neutrophil numbers. In embodiments, the reference is a control value. In embodiments, the reference is the leukocyte and neutrophil numbers in a sample from a healthy person.

In embodiments, the method decreases the rate of growth of neutrophil to lymphocyte ratio (NLR) compared to a reference. In embodiments, the reference is a control value. In embodiments, the reference is the rate of growth of neutrophil to lymphocyte ratio (NLR) in a sample from a healthy person.

In embodiments, no intravenous vitamin C is administered for a recovery period after administration of one or more pulse dose and lower dose (press dose). In embodiments, the recovery period is one day to one week. In embodiments, the recovery period is one day. In embodiments, the recovery period is two days. In embodiments, the recovery period is three days. In embodiments, the recovery period is four days. In embodiments, the recovery period is five days. In embodiments, the recovery period is six days. In embodiments, the recovery period is one week. In embodiments, the recovery period is two weeks. In embodiments, the recovery period is three weeks. In embodiments, the recovery period is one month. In embodiments, the recovery period is two months. In embodiments, the recovery period is three months. In embodiments, the recovery period is greater than 3 months.

In embodiments, the pulse dose and lower dose (press dose) are administered over a treatment cycle, where the treatment cycle is between one week and 5 weeks. In embodiments, the treatment cycle is between one week and 4 weeks. In embodiments, the treatment cycle is between one week and 3 weeks. In embodiments, the treatment cycle is between one week and 2 weeks. In embodiments, the treatment cycle is about 2 weeks.

In embodiments, the treatment cycle includes a recovery period after administration of one or more pulse dose and lower dose (press dose). In embodiments, the recovery period is one day to one week. In embodiments, the recovery period is one day. In embodiments, the recovery period is two days. In embodiments, the recovery period is three days. In embodiments, the recovery period is four days. In embodiments, the recovery period is five days. In embodiments, the recovery period is six days. In embodiments, the recovery period is one week. In embodiments, the recovery period is two weeks. In embodiments, the recovery period is three weeks. In embodiments, the recovery period is one month. In embodiments, the recovery period is two months. In embodiments, the recovery period is three months. In embodiments, the recovery period is greater than 3 months.

In embodiments, the treatment cycle is repeated one or more times. The number of treatment cycles can be determined by the skilled clinician. In embodiments, the doses and/or timing of subsequent treatment cycle(s) are the same as the initial treatment cycle. In embodiments, the doses and/or timing of subsequent treatment cycle(s) differ from those of the initial treatment cycle.

### EXAMPLES

### Example 1: Pulse-Press Intravenous Vitamin C Therapy

Intravenous vitamin C therapy is given by intermittent, high dose infusion that involves administering high-dose vitamin C (15-100 grams) over a 1-2 hour window and repeated twice weekly (every 2-4 days). The intermittent frequency of high-dose administration is referred to as a PULSED approach or as a PULSE dose.

As an adjunct to high-dose intermittent vitamin C infusions, experiments described herein have led to the developments of a method for administering continuous low to mid doses of vitamin C infusion using an elastomeric pump (non-electric) infusion pump over an 8 to 48 hour window. This method of continuous infusion targets a therapeutic dose anywhere between 4 to 50 grams of vitamin C per infusion window and is referred to as a PRESS approach or a PRESS dose.

The intravenous vitamin C+(IVC+) protocol for the treatment of cancer, and other chronic conditions, described herein combines the pulse approach with a press protocol into a PULSE-PRESS approach whereby vitamin C infusions alternate between a high-dose rapid infusion with a low to mid dose continuous infusion.

The PULSE is short-acting and initiates a high amount of oxidative stress in cancer cells and the PRESS is long-acting and creates longer continuous stress on the cells. This persistent stress on cancer cells prevents the stronger surviving cells from recovering from the initial PULSE dose and reduces or inhibits the development of resistance.

The application of pulse-press is a breakthrough strategy of the IVC+ Plan. The intermittent, high-dose bolus of IVC delivers a powerful “pulse” against the tumor cells. This pulse is immediately followed by a lower and slower dose of IVC administered by pump over a 36-hour period. This follow-up strategy maintains the “press” of constant oxidative stress on ALL of the dying cancer cells, thus disallowing strong cell recovery.

Table 1 outlines the use of the PULSE-PRESS approach for the administration of IVC+ Protocol in the clinical setting. This chart represents a frequency of infusion administration under the protocol and represents just one of many possible combinations of this approach that can be tailored by medical doctors to meet the treatment needs of the patient.

TABLE 1
Example PULSE-PRESS Protocol

| Monday | Tuesday | Wednesday | Thursday | Friday | Sat/Sun |
|---|---|---|---|---|---|
| Week 1 | | | | | |
| IVC Pulse | IVC | IVC Pulse | IVC | IVC Pulse | Rest & |
| (15 gm) | Press | (25 gm) | Press | (50 gm) | Recover |
| followed by | (0.5 | followed by | (0.5 | | |
| IVC Press | gm/hr) | IVC Press | gm/hr) | | |
| (0.5 gm/hr) | | (0.5 gm/hr) | | | |
| Week 2 | | | | | |
| IVC Pulse | IVC | IVC Pulse | IVC | IVC Pulse | Rest & |
| (50 gm) | Press | (50 gm) | Press | (50 gm) | Recover |
| followed by | (0.5 | followed by | (0.5 | | |
| IVC Press | gm/hr) | IVC Press | gm/hr) | | |
| (0.5 gm/hr) | | (0.5 gm/hr) | | | |

This clinical application of the IVC+ Protocol is centered on a 2-day cycle of a rapid PULSE infusion immediately followed by a PRESS continuous infusion at a rate of 0.5 mg of Vitamin C per hour. Over the two week period, each pulse dose of vitamin C is increased to a therapeutic dose as outlined in the original Riordan IVC Protocol [See for example. Ref 1]. Each week back to back pulse-press doses are given followed by a final pulse dose leading into a rest period. These cyclic rest periods are important wherein they provide recovery time necessary for the patient to assimilate the oxidative stress generated within the body.

After the 2-week treatment cycle and resting period, the patient's progress is assessed by the medical doctor who coordinates with the patient whether to adjust and/or repeat the treatment cycle.

### Example 2 Updated Analysis of Pilot Clinical Study

Intravenous vitamin C (IVC) therapy is widely used in naturopathic and integrative oncology. Preclinical studies of large doses of ascorbic acid (vitamin C) have been reported to show significant anti-cancer effects in animal models and tissue culture investigations.

Studies on understanding the biological activities of ascorbate have led to a number of hypotheses for mechanisms of anti-cancer activity, such as the generation of significant quantities of hydrogen peroxide by the autoxidation of pharmacological concentrations of ascorbate, changes in the metabolic activity, and stimulation of the enzymes that have a cofactor requirement for ascorbate. In addition, high dose ascorbic acid may improve the anti-cancer action of chemotherapeutic agents, boost immune cell functioning, and inhibit angiogenesis.

Many case studies demonstrated the effectiveness of intravenous vitamin C, with varying degrees of success. Clinically published IVC case studies report efficacy against a variety of cancers in humans, including pancreatic cancer, bone metastases accompanying breast cancer, non-Hodgkin's lymphoma, liver carcinoma, colon carcinoma, and ovarian cancer.

Several Phase I and Phase II clinical trials have been conducted in the last ten years to test safety and efficacy when IVC is used as an adjuvant with chemotherapy. The results of these trials confirm that IVC can be administered safely.

Most practitioners administer IV ascorbate to cancer patients by bolus infusions 2-3 times per week.

There have been two clinical trials that used continuous IVC infusions. Cameron and Pauling performed a clinical trial in 100 terminal cancer patients. The protocol included an initial 10 day course of IV ascorbate, at a relatively low daily dose of 10 g/day given by continuous infusions, followed by daily oral intakes of 10-30 g/day, in divided doses. Their results showed increased survival time and improved quality of life of the patients, compared with patients who had not received IVC. [See for example Ref 3]

The ideas of Linus Pauling were extended in the model developed by Dr. Hickey. He described “The dynamic flow model” that proposes restoring human physiology by administering excess ascorbate, over and above the amount normally absorbed, spread throughout the day, so a consistent supply is achieved.

The second trial of the treatment of cancer patients by continuous infusions was conducted by Dr. Riordan. In this Phase I clinical trial (hereafter referred to as the Riordan trial), patients were administered continuous infusions using an infusion pump [See for example Ref 1]. In the Riordan Clinic trial, patients were treated by continuous infusion, which was administered over much longer periods of time than bolus intermittent treatments. For most patients, the duration of the continuous infusion was at least 20 hours, as the duration of bolus infusion is from one hour to three hours, depending on the dosage.

The trial lasted eight weeks and involved terminal patients with poor prognosis. Twenty-four (24) subjects were given continuous IVC at doses between 150 and 710 mg/kg/day (10-50 grams per day). Most of the patients had colon cancer with liver and lung metastasis and three patients had pancreatic or liver cancer. All patients had several chemotherapy/radiation treatments before entering the study. Seventy-none percent (79%) of the patients had a metastatic tumor.

Recently, previously unpublished parameters from the clinical study were analyzed, including blood chemistry and blood count parameters that are reportedly related to patient prognosis and degree of inflammation [See for example Ref 2]. This included: absolute neutrophil and lymphocyte counts and the neutrophil-to-lymphocyte ratio; lactate dehydrogenase, an enzyme involved in tumor initiation, metastasis, and recurrence; creatinine, the depletion of which is associated with cachexia; and glucose, as hyperglycemia is common in cancer patients.

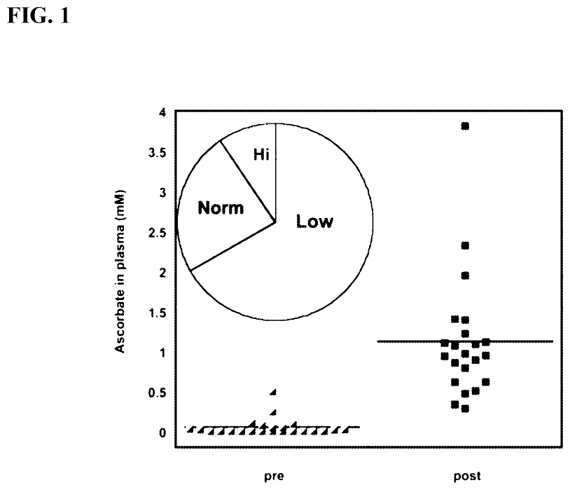

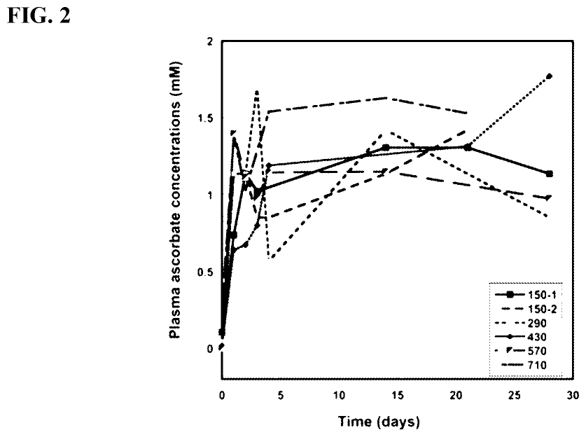

The most obvious effect of IVC therapy was to increase patient vitamin C levels. Consistent with other reports, the plasma ascorbate measurements conducted in the Riordan trial showed that vitamin C depletion in cancer patients is common. In fact, ten out of twenty-four subjects entered the trial with plasma ascorbate concentrations undetectable by the colorimetric ascorbate assay used at that time, with another four having ascorbate concentrations below the normal range (FIG. 1). IVC infusion increased plasma levels to 1 mM. (FIG. 2) This likely replenished depleted tissue ascorbate stores as well.

As lymphocytes and neutrophils have important roles in tumorigenesis and carcinogenesis, the effect of the treatment on these parameters was analyzed. As the result of chemotherapy, neutrophil and lymphocyte counts typically decrease in cancer patients, with the effect being more severe for lymphocytes.

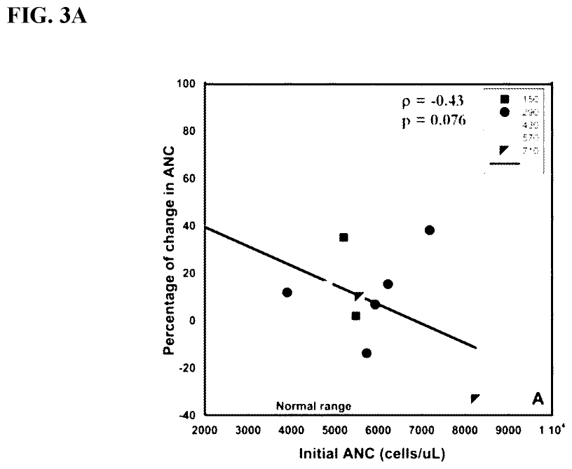

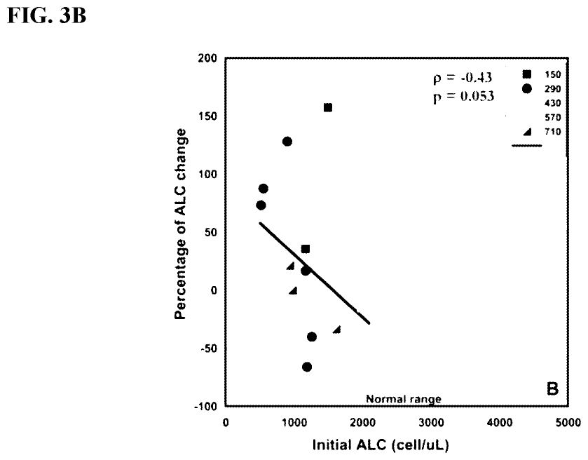

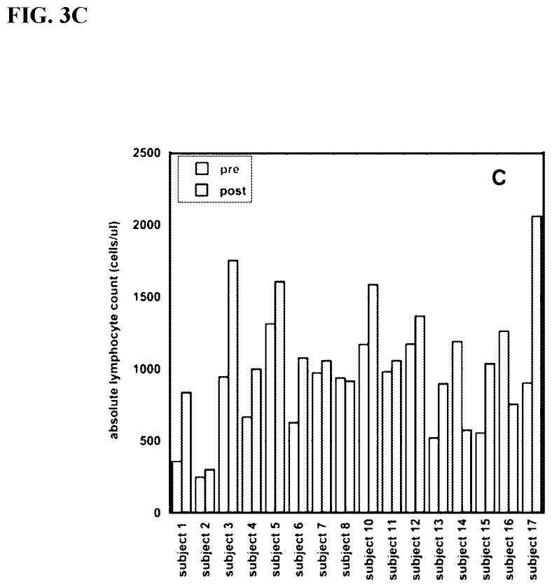

Analysis of white blood cell counts for patients in the Riordan trial indicated the potential for IVC to increase lymphocyte and neutrophil counts for patients in whom these numbers are below normal, while reducing neutrophil counts in patients for whom neutrophil counts are elevated. (FIG. 3)

It was particularly important for lymphocyte counts. Lymphopenia commonly occurs in cancer patients who had chemotherapy and high levels of oxidative stress induced by treatment, predicting a poor prognosis.

In the study population, about half of the patients who started intervention had lymphocyte counts lower than normal range. For patients with severe lymphopenia, who completed treatment, the median improvement in the lymphocyte count was 69%, and for all patients with lymphocyte levels lower than normal range the median improvement was 22%. These data proved that continuous IVC can improve immune function of cancer patients by increasing the level of lymphocytes, especially in patients with low lymphocyte count. The data also indicated that lower doses are more favorable for the improvement of lymphocyte count.

As absolute neutrophil counts and neutrophil-to-lymphocyte (NLR) ratios are useful prognostic factors in a variety of cancers, with higher values of NLR indicating lower survival times, the effect of continuous infusion on these parameters was analyzed. For cancer patients in general, increased neutrophil counts are consistent with systemic inflammation, and a neutrophilic response is associated with poor prognosis, as it can inhibit the immune system by suppressing the cytotoxic activity of T cells. For most of the patients, the tendency during treatment was for normalization of the neutrophil counts, i.e. improvement of neutrophil counts at the low level of this parameter and a decrease for the higher values.

The present analysis of neutrophil-to-lymphocyte ratios (NLR) also demonstrated the regulatory effect of IVC. NLR has been used to assess inflammatory response and has been suggested as a prognostic factor in a variety of cancers. In particular, cut-off values ranging between 2.0 and 4.0 were associated with a significant increase in all-cause mortality. As NLR may reflect the balance between the activation of the inflammatory pathway and the anti-tumor immune function, elevated NLR due to neutrophilia is linked to accelerated tumor development.

In the updated analysis, most of the patients entered the trial with NLRs well above this cut-off. Continuous IVC therapy tended to decrease the rate of growth of NLR. Moreover, the predictive potential of NLR was confirmed. The data demonstrated the relationship between the survival of patients and the rate of growth of NLR, as NLR increases correlated with lower survival times of the patients.

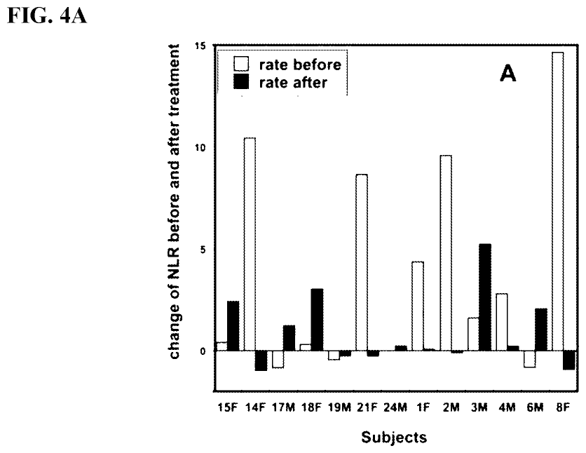

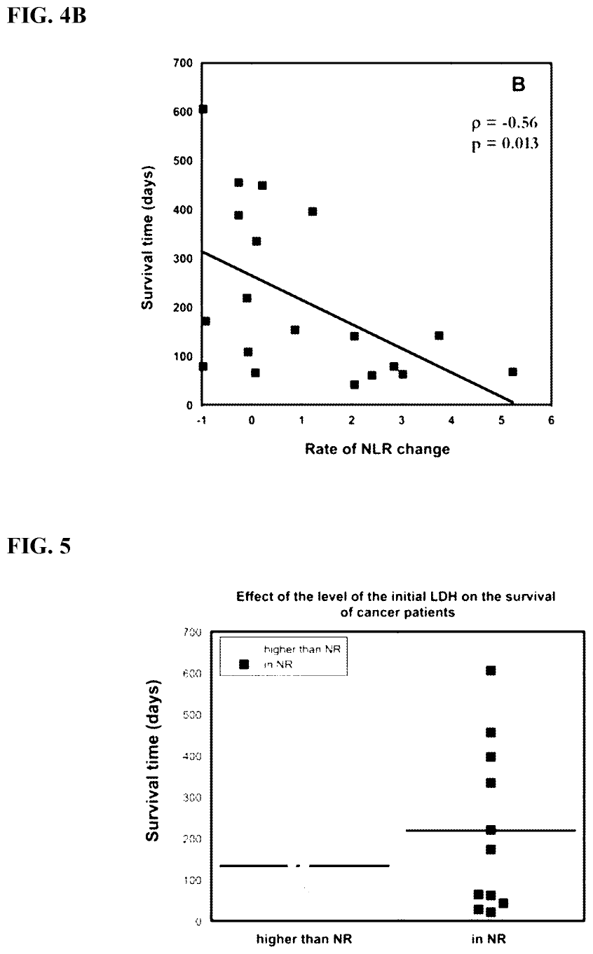

The rate of change in this ratio (ΔNLR) was examined for each patient before and after therapy (FIG. 4). The comparison of the trend in the change of NLR measured for periods one week before treatment and during treatment demonstrated that the rate of change was decreased. This suggests that IVC may reduce NLR levels, thus improving prognosis. Since the rate of increase in NLR for patients with initially elevated values decreased during IVC therapy, ascorbate may be decreasing inflammation in these subjects.

As activation of glycolytic metabolism is a significant characteristic of tumor cells, and since lactate dehydrogenase (LDH) is an important coenzyme in glycolysis, elevated levels of serum lactate dehydrogenase was thought to be a useful prognostic biomarkers. Lactate dehydrogenase is elevated in many types of cancers; it has been linked to tumor growth, maintenance and invasion.

The rate of increase of LDH was calculated before and after treatment (FIG. 5). The value of this parameter (LDH rate of growth) was decreased in 38% of the patients, increased in 28.6%, and was not changed in 33.4% of patients. The result that LDH decreased in 38% of the subjects is remarkable, considering their illness.

The median survival time for the all participants with initial LDH higher than normal range (LDH>245 U/L) was 95 days. In contrast, the median survival time for all subjects with normal initial LDH values was 173 days.

Hyperglycemia is another prognostic factor in cancer patients. It is common in cancer patients and represents a challenge during therapy. For example, about 70% of pancreatic cancer patients have impaired glucose tolerance. Moreover, there is a link between the lowering of blood glucose concentration and remission of malignancy. In one study, patients under insulin coma therapy for six months (for psychosis) were reported to become free of large tumor burdens considered incurable by their oncologists. (See for example Refs. 4-5).

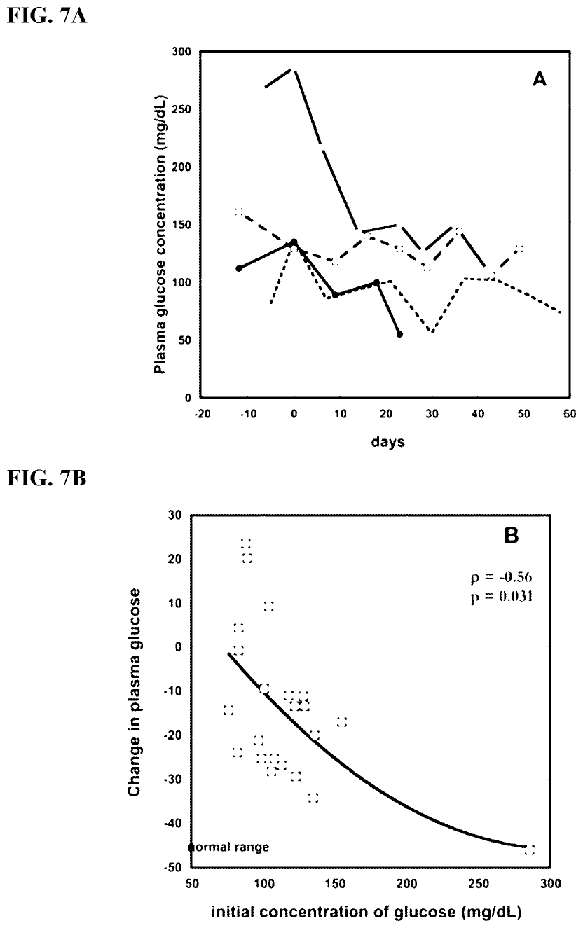

Hyperglycemia was common in the cancer patients. Two thirds of the patients in our study had above normal blood glucose concentrations. There were changes in blood glucose during IVC therapy. Blood glucose levels were decreased for patients with the concentrations higher than normal range during IVC therapy (FIG. 7).

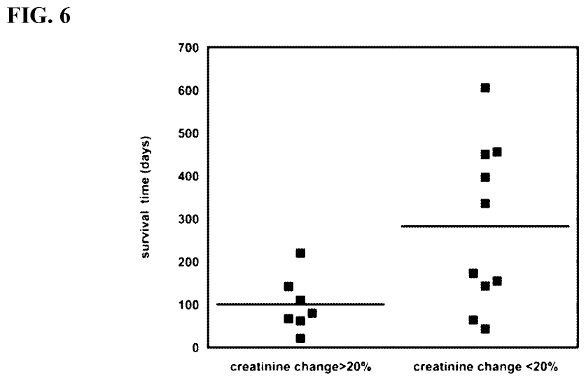

Several clinical trials have established that IVC can be administered safely. In the continuous IVC infusion trial (See Ref 1), from which data for the analysis herein were obtained, side effects were mostly minor, and the criterion for stopping the clinical trial (two or more Grade 3 or higher adverse events at a given dose at least possibly related to the treatment) was never reached. Briefly, blood chemistry parameters that serve as indicators of renal function (BUN, creatinine, and uric acid) remained relatively stable, or, in the case of uric acid, decreased during therapy. Only four subjects experienced BUN increases during therapy (FIG. 6).

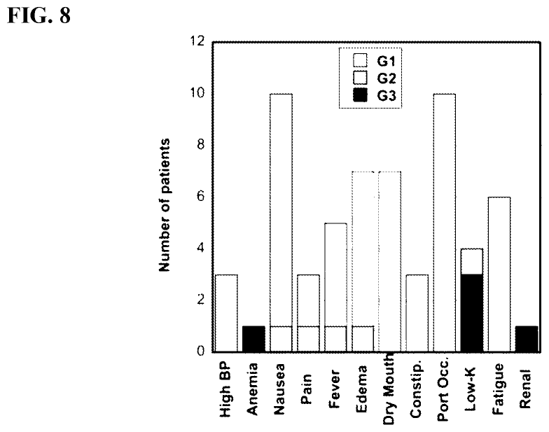

The most common side effects were nausea (11 subjects), injector port occlusion (10 subjects), dry skin or mouth (7 subjects), edema (7 subjects) and fatigue (6 subjects). These were generally minor (Grade 1). Most of the Grade 3 events involved hypokalemia, which is considered possibly related to the ascorbate therapy. (FIG. 8)

In summary, the updated analysis demonstrated the regulatory and normalizing effect of continuous IVC infusions on lymphopenia, neutrophil-to-lymphocyte ratios, and absolute neutrophil counts. Despite the very poor health status of patients, continuous IVC treatment had positive effects on the important parameter that characterized tumor metabolism (lactate dehydrogenase) and blood glucose concentration.

### Example 3: Treatment of Patients Using Pulse-Press Intravenous Vitamin C Therapy

The protocol has been used on about six subjects to ascertain safety, feasibility and usability of the pump and in coordinating the process with existing therapies. The types of cancer for these patients varied. Results showed the pulse press protocol was well tolerated.

Intravenous vitamin C therapy was administered to a patient suffering from cancer. The therapy was well-tolerated and the patient surprising showed no signs of the development of chemo resistance. The treatment appeared efficacious.

### REFERENCES

### Each of the Following References is Incorporated Herein by Reference in its Entirety for all of its Methodologies, Compositions of Matter, Devices, Materials, Etc

1. Riordan H D et al. P R Health Sci J. 2005 December; 24(4):269-76.
2. Nina Mikirova, Joseph Casciari, Ronald Hunninghake. Continuous intravenous vitamin C in the cancer treatment: reevaluation of a Phase I clinical study. Functional Foods in Health and Disease 2019; 9(3): 180-204.
3. Cameron E, Pauling L., Proc Natl Acad Sci USA. 1976 October; 73(10):3685-9.
4. Koroljow S. Insulin coma therapy. PsychiatrQ 1962, 36, 261-270.
5. Krone C A, Ely J T A. Controlling hyperglycemia as an adjunct to cancer therapy. Integer. Cancer Ther 2005, 4, 25-31.

What is claimed is:

1. A method for reducing chemotherapeutic resistance in a cancer patient, the method comprising: administering a pulse dose of vitamin C and subsequently administering a continuous perfusion of a lower dose of vitamin C, wherein the continuous perfusion is administered over an infusion window.

2. The method of claim 1, wherein the pulse dose is administered over time of about 1 to about 2 hours.

3. The method of any one of claims 1-2, wherein the pulse dose is repeated at least twice weekly.

4. The method of any one of claims 1-3, wherein the pulse dose is administered intravenously.

5. The method of any one of claims 1-4, wherein the infusion window is about 8 to about 48 hours.

6. The method of any one of claims 1-5, wherein the continuous perfusion is administered using an elastomeric pump.

7. The method of any one of claims 1-6, wherein the pulse dose is between 15 grams and 100 grams vitamin C.

8. The method of any one of claims 1-7, wherein the lower dose is between 4 grams and 50 grams vitamin C per infusion window.

9. The method of any one of claims 1-8, wherein the lower dose is administered up to seven days per week.

10. The method of claim 9, wherein the lower dose is administered up to five days per week.

11. A method of treating cancer in a subject having cancer, the method comprising: administering a pulse dose of vitamin C and subsequently administering a continuous perfusion of a lower dose of vitamin C.

12. The method of claim 11, wherein the pulse dose is administered over time of about 1 to about 2 hours.

13. The method of any one of claims 11-12, wherein the pulse dose is repeated twice weekly.

14. The method of any one of claims 11-13, wherein the pulse dose is administered intravenously.

15. The method of any one of claims 11-14, wherein the continuous perfusion is administered over an infusion window of about 8 to about 48 hours.

16. The method of any one of claims 11-15, wherein the continuous perfusion is administered using an elastomeric pump.

17. The method of any one of claims 11-16, wherein the pulse dose is between 15 grams and 100 grams vitamin C.

18. The method of any one of claims 11-17, wherein the lower dose is between 4 grams and 50 grams vitamin C per infusion window.

19. The method of any one of claims 11-18, wherein the lower dose is administered up to seven days per week.

20. The method of claim 19, wherein the lower dose is administered up to five days per week.

21. A method of treating cancer in a subject having cancer, the method comprising: administering a high dose of vitamin C wherein the high dose comprises from about 15 grams to about 50 grams vitamin C every other day and subsequent to each high dose, administering a continuous perfusion of a lower dose of vitamin C wherein the lower dose is administered by continuous perfusion over an infusion window of about 8 to about 48 hours at a rate of about 0.5 grams vitamin C per hour.

22. The method of claim 21, wherein the pulse dose and lower dose (press dose) are administered over a treatment cycle.

23. The method of claim 22, where the treatment cycle is between one week and 5 weeks.

24. The method of any one of claims 22-23, wherein the treatment cycle comprises a recovery period after administration of one or more pulse dose and lower dose.

25. The method of any one of claims 22-24, wherein the high dose is administered three times in a seven day period.

26. A method of treating a subject in need thereof, the method comprising administering a pulse dose of vitamin C and subsequently administering a continuous perfusion of a lower dose of vitamin C, wherein the continuous perfusion is administered over an infusion window.

27. The method of claim 26, wherein the subject has leukocyte and neutrophil numbers are below normal compared to a reference and wherein the method increases leukocyte and neutrophil numbers.

28. The method of claim 26, wherein the method decreases the rate of growth of neutrophil to lymphocyte ratio (NLR) compared to a reference.

29. The method of claim 26, wherein the subject has an elevated lactate dehydrogenase (LDH) level compared to a reference and the method reduces lactate dehydrogenase (LDH) level.
